(19) United States
(12) Patent Application Publication
(10) Pub. No.: US 2007/0238145 A1
(43) Pub. Date: Oct. 11, 2007

(54) Phenotypic engineering of spores

(76) Inventors: Mindy A. Cote, Woonsocket, RI (US); Linda Ferencko, North Kingstown, RI (US); M. Boris Rotman, Jamestown, RI (US)

Correspondence Address: Bruce F. Jacobs;Jacobs Patent Office, P.O. Box 390438, Cambridge, MA, 02139, US

(21) Appl. No.: 11/708,829

(22) Filed: Feb. 21, 2007

Related U.S. Application Data
(60) Provisional application No. 60/775,252, filed on Feb. 21, 2006.

(51) Int. Cl. C12Q 1/02; C12N 1/21

(52) U.S. Cl. 435/29; 435/243

(57) ABSTRACT

The biological functionality of living microbial spores is modified using phenotypic engineering to endow the resulting modified spores with novel functionality that extends the usefulness of the spores for a variety of practical applications including, for example, sterility testing, the release of active compounds, and cell-based biosensing systems. A preferred embodiment entails engineering Bacillus spores to acquire synthetic new functions that enable the modified spores to sense and rapidly transduce specific germination signals in their surroundings. The newly acquired functions allow the spores to perform, for example, as self-reporters of cellular viability, self-indicating components of cell-based biosensors, and in other analytical systems.

### BACKGROUND OF THE INVENTION

This invention is directed to the phenotypic engineering of spores, particularly to the preparation of modified spores useful in the fields of biological and biochemical indicators, most particularly those used for a variety of assays including bio-sensing and sterility testing.

More particularly this invention is directed to phenotypically engineered spore that includes a man-made functionality under the control of the spore's natural germination apparatus to give the spore self-reporting capability. The man-made functionality is introduced by contacting the spores with a hydrophobic compound. Suitable such functionalities preferably include fluorogenicity, chromogenicity, chemiluminogenicity, bioluminogenicity, and indigogenicity.

Most particularly, this invention relates to novel methodologies that utilize phenotypic engineering to modify the performance of living spores as rapid and rugged indicators of environmental changes. An example of such methodologies is the phenotypic engineering of living Bacillus spores to create a new function enabling the spores to perform as fluorogenic biological microorganisms. The new fluorogenic functionality is advantageous for determining susceptibility of microbial spores to sterilization conditions and other chemical and physical treatments.

Sterility Testing

In many industries, sterilization processes are routinely used to kill micro-organisms that may contaminate food, beverages, solutions, equipment or devices. Different techniques may be used for sterilizing including steam autoclaving for about 10 to 60 minutes at temperatures ranging from about 110° C. to 132° C., dry heating for 30 or more minutes at 150° C. to 160° C., and exposure to radiation or chemicals such as ethylene oxide, hydrogen peroxide, and peracetic acid.

For most processes, it is critical to monitor the effectiveness of the equipment and procedure used for sterilizing. For example, it is standard practice in medical and pharmaceutical institutions to use an indicator for sterility assurance to ascertain that no living microorganisms are present in materials that have undergone a sterilization process. Over the years, different types of sterility indicators have been developed including biological and chemical indicators.

While chemical indicators are often used to monitor gross failures of sterilization processes, it is well recognized in the art of sterilization that biological indicators consisting of living microbial spores are one of the most accurate and reliable systems for sterility assurance. Microbial spores are preferred over vegetative cells because spores are more resistant to physical and chemical treatments. A traditional method for sterility testing is to place a carrier with spores near the items to be sterilized, and after sterilization, to detect any surviving spores by incubating the spores in a bacteriological growth medium. Spore outgrowth after incubation periods ranging from one to seven days is taken as an indicator of inadequate sterilization. A major disadvantage associated with this method is that seemingly sterilized articles must be stored for prolonged times until test results become available.

In the last two decades, efforts to develop faster methods for monitoring sterility have been directed at techniques in which bacterial enzymes, either present in or extracted from vegetative cells, are substituted for the traditional biological indicators based on outgrowth of microbial spores. For example, an enzyme-based sterility indicator is disclosed in U.S. Pat. No. 5,073,488 (Matner et al.) and indicator systems using several different enzymes and their respective substrates have also been described in U.S. Pat. No. 5,486,459 (Burnham et al.). Typically, in the enzyme-based technology, a carrier with a particular enzymatic activity is placed near the items to be sterilized, and after sterilization, the remaining enzymatic activity is determined by incubating the indicator with a specific substrate yielding detectable product(s). The amount of remaining enzymatic activity is used as a parameter to assess the efficacy of the sterilization process. Thus, the reliability of this type of enzyme-based indicators hinges on the implicit assumption that the rate of enzyme inactivation correlates accurately with the rate of spore killing. Consequently, using this type of indicator, inadequate sterilization is indicated by partial enzyme inactivation or no enzyme inactivation. However, and most importantly, complete enzyme inactivation is not a reliable sterility assurance test because enzymes may be prematurely inactivated in comparison to spore killing. Diverse efforts to circumvent the problem of premature enzyme-inactivation have been described. For example, spores or the source of active enzyme may be chemically treated to enhance the resistance of the enzyme to premature inactivation as described in U.S. Pat. No. 7,045,343 (Witcher et al.). The chemicals described in that patent typically include surfactants, waxes, and oils such as polyglycerol alkyl esters and ethoxylated glycerol esters.

For these reasons, enzyme-based indicators do not provide the same type of sterility assurance obtained with traditional indicators based on measuring outgrowth of surviving spores. In this respect, enzyme-based indicators resemble chemical indicators in that both can only indicate gross failures of the sterilization equipment or process.

Another drawback of enzyme-based indicators is that the amount of enzyme present in the indicator system has to be carefully calibrated to ensure that the rate of enzyme inactivation in fact correlates with the rate of spore killing. However, calibrating enzymatic activity is not a simple procedure since it depends on a number of parameters such as enzyme concentration, enzyme purity, and incubation temperature. The problems associated with calibrating enzymatic activity are compounded when using either crude enzyme preparations or microbial spore preparations that usually contain relatively large concentrations of enzymes from vegetative cells contaminating the preparations. For example, preparations of G. stearothermophilus spores are normally contaminated with 5-20% of vegetative cells.

In efforts to circumvent the aforementioned problems associated with enzyme-based indicators, dual systems have been recently introduced in which an enzyme-based indicator for early warning is used together with a traditional indicator based on spore outgrowth. For example, an invention using a dual system is disclosed in U.S. Pat. No. 5,418,1670 (Matner et al.) which describes a sterility indicator that contains in separate compartments a strip with Geobacillus stearothermophilus spores that have detectable alpha-glucosidase activity; growth medium; and 4-methylumbelliferyl-alpha-D-glucoside, a fluorogenic substrate of alpha-glucosidase. After sterilization, the spores, the growth medium, and the substrate are mixed and incubated. Following 2-4 hours of incubation, the presence of alpha-glucosidase activity (detected by an increase in fluorescence) indicates inadequate sterilization. On the other hand, if enzymatic activity is undetectable after four hours of incubation, the indicator is further incubated for several days in order to detect outgrowth of any surviving spores. Consequently, this type of combination indicator system does not represent an improvement over traditional biological indicators since it still requires several days to provide reliable sterility assurance.

Another type of enzyme-based sterility indicator is disclosed in U.S. Pat. No. 5,770,393 (Dalmasso et al.). It uses enzyme production during outgrowth of surviving spores as a method to increase assay sensitivity and thereby reduce assay time. For example, alpha-amylase activity produced by vegetative cells is indicative of spore outgrowth in the indicator and may be detected after 2-8 hours of incubation using a specific alpha-amylase substrate. This type of indicator system, however, does not have the single-spore sensitivity of conventional biological indicators based on measuring spore killing by spore outgrowth.

Although it is traditional to monitor sterilization processes using spore outgrowth as the “viability parameter,” other cellular activities closely related to spore viability have also been used as parameters of cell survival. For example, U.S. Pat. No. 5,795,730 (Tautvydas) discloses certain biological reactions, such as loss of refractivity occurring during spore germination, may be used to measure the effectiveness of sterilization processes. Spore germination is a complex, irreversible process consisting of many different biochemical reactions triggered when microbial spores encounter outgrowth conditions. Germination is independent of transcriptional control and includes three sequential stages: (I) spore activation; (ii) initiation of germination; and (iii) spore outgrowth (T. S. Stuart, Microbiology (1998) p. 34). Spore activation takes place when a germinant receptor (e.g., L-alanine receptor) that is also a protease is activated by heat or one of several chemicals. The second stage, initiation, ensues when the activated spore encounters a germinant (e.g., amino acids, adenosine, and glucose). It is during initiation that the spore undergoes irreversible changes including increased outer coat permeability that allow both influx of nutrients and water into the cell and efflux of cellular components. In addition, some time during initiation the spore loses its heat resistance and refractivity. The outgrowth stage is characterized by spores returning to their vegetative cell morphology and functions. In contrast to the outgrowth stage which necessitates de novo synthesized cellular components, both the first and second stages of germination use only preformed components. Since germination is a vital process preceding spore outgrowth, sterilization conditions resulting in complete loss of a spore's ability to germinate will generally indicate adequate sterilization. A commonly used method to determine germination in a spore suspension is based on loss of light scattering properties due to biochemical changes in the spore's wall. U.S. Pat. No. 5,795,730 (Tautvydas) discloses a method to rapidly measure the effectiveness of sterilization processes by determining the rate of spore germination after sterilization using a loss of light scattering as the parameter. The drawbacks of this method are that measurements of light scattering requires expensive instrumentation, and also that the sensitivity of the method is considerably lower than that of traditional testing by spore outgrowth.

The present invention discloses novel biological indicator systems for sterility assurance based on phenotypic engineered spores that have capabilities as self-reporters of germination. Therefore, the engineered spores function more efficiently than normal spores currently used as biological indicators for sterility testing.

Cell-based Biosensing

Living microbial spores have been previously used as sensing components in devices for detecting and identifying pathogenic bacterial cells, macromolecules and other analytes directly from a test sample. In these systems, the spores were used to sense specific signals from analytes and respond to them by establishing an analyte-independent signal amplification system. For example, U.S. Pat. No. 6,596,496 (Rotman) discloses methodologies that provide a particularly efficient technique to conduct thousands of parallel assays in an array of microscopic biosensors. These methodologies teach a label-free (label-less), growth-independent, analytical system (termed “LEXSAS™”) using enzyme-free spores for rapid detection and identification of different analytes directly from a test sample. In that invention, the test material is mixed with a germinogenic source and enzyme-free spores prepared from selected bacterial strains. The mixture stands for a short time to allow for analyte-induced spore germination and subsequent de novo synthesis of an enzyme capable of producing a germinant in the presence of the germinogenic source. The germinant promotes further spore germination with concomitant de novo enzyme synthesis that results in a propagating cascade of analyte-independent germination. The end point of the cascade can be measured using an assortment of physical and enzymatic parameters, e.g., chromogenic or fluorogenic substrates.

The present invention serves to improve previously developed biosensors by utilizing phenotypic engineered spores that have self-reporting capabilities and therefore can function more efficiently than the previous spores that have been used in various biosensing devices.

Spores have previously been genetically engineered to produce an immune response to an antigen, c.f. U.S. Pat. No. 5,800,821 (Acheson et al.), which discloses a method of stimulating a vertebrate animal to produce an immune response to at least one antigen. The method includes genetically engineering a bacterial cell with DNA encoding at least one antigen and inducing the bacterial cell to sporulate, then orally administering the bacterial spores to an animal. The bacterial spores germinate in the gastro-intestinal tract of the animal and express the antigen so that it comes into contact with the animal's immune system and elicits an immune response.

U.S. Pat. No. 5,766,914 (Deits) discloses a method of producing and purifying an enzyme by selecting a spore forming host organism, preparing a genetic construct consisting of a DNA sequence encoding a desired enzyme and a DNA sequence directing synthesis of the desired enzyme during sporulation, inserting the genetic construct into the host organism, culturing the transformed host organism under sporulating conditions to obtain host organism spores with the enzyme integrally associated to the spores, and then treating the host organism and enzyme combination to remove any impurities, if necessary. The free enzyme can be obtained by cleaving the connection between the host organism and the enzyme. The combination of the enzyme and host organism is both a stabilized and an immobilized enzyme preparation.

### SUMMARY OF THE INVENTION

The present invention is directed to procedures, devices and kits for engineering living spores for the purpose of creating phenotypically engineered spores so as to have man-made functionalities not previously observed in nature. The invention chemically manipulates spores as hydrophobic, inert particles suspended in organic solvents maintaining their ability to germinate as normal spores.

More particularly, the present invention is directed to phenotypically engineered spores that includes a man-made functionality under the control of the spore's natural germination apparatus to give the spore self-reporting capability. The man-made functionality is introduced by contacting the spores with a hydrophobic compound which has a visual generating property such as fluorogenicity, chromogenicity, chemilumino-genicity, bioluminogenicity, and indigogenicity.

The invention makes available different embodiments to obtain engineered spores useful for sterility testing and for delivering signals that can be used for detecting and identifying particulate analytes such as microbial cells, viruses, and biological macro-molecules such as antibodies, cytokines, nucleic acids (DNA and RNA) and proteins.

### DESCRIPTION OF THE PREFERRED EMBODIMENTS

The present invention relates to the preparation and practical applications of phenotypic engineered spores in which a man-made functionality has been introduced and placed under control of the spore's natural germination apparatus.

This invention further relates to sterility testing utilizing the phenotypic engineered spores as self-indicators of adequate sterilization conditions. Preferably, the man-made functionality of these spores is chromogenic or fluorogenic.

The invention further relates to biosensing to detect analytes through the use of phenotypic engineered microbial spores acting as both signal-sensors and signal-transducers of analyte-specific signals. An analyte is detected by placing a sample suspected of containing the analyte in a mixture of phenotypic engineered spores and a germinogenic source. The end point is a detectable signal, preferably bioluminescence, color, or fluorescence that can be used to determine the presence, location, and number of discrete entities of analytes.

This invention further relates to test kits containing the phenotypic engineered spores.

Generally, the phenotypically engineered spores of this invention are produced by suspending living spores in a liquid, contacting the suspended spores with a hydrophobic compound under conditions which cause the hydrophobic compound to incorporate and self-assemble into the spores to form modified spores, and recovering the modified spores.

More particularly, in a first embodiment of this invention, the phenotypic engineered spores are prepared from dried living spores containing less than about 5% extracellular water. The dried spores are suspended in a non-aqueous solution containing a selected hydrophobic molecular probe similar to those listed in Table 1. The resulting spore suspension is incubated for a sufficient period of time to allow incorporation and self-assembling of the selected hydrophobic molecular probe in the spores. Finally, the organic solvent is removed, preferably under vacuum.

The living spores engineered according to this method not only remain viable, but also become self-reporters of germination. Accordingly, the engineered spores are suitable for using as direct biological indicators or as components of cell-based biosensing devices.

The dried spore preparation (before engineering) may be prepared by different well known procedures. A typical procedure entails heat-activating a spore suspension in sterile deionized water at a temperature of about 50 to 110° for about 5 to 60 minutes, for example, 65° C. for about 30 minutes, and then spinning the suspension at 10,000×g for about 5 minutes to pellet the spores and form a supernatant. After removal of the supernatant, the pellets can be dried under vacuum for about 90 to 120 minutes over a desiccant such as silica gel. The dried spores should contain less than about 5% extracellular water, preferably less than about 1%.

Appropriate organic solvents for preparing the non-aqueous suspensions include chemicals such as acetone, acetonitrile, ethyl acetate, methyl ethyl ketone, tetrahydrofuran, and toluene. The spore suspension may be formed by pipetting up-and-down the dried spores with the non-aqueous solution containing the selected molecular probe to be engineered into the spores. The engineered spores using this methodology were experimentally shown to have acquired a man-made function controlled by the spore's innate germination apparatus. This unexpected result probably stems from the fact that the hydrophobic molecular probes self-assemble forming a discrete boundary around the spore's outer coat (as determined by ultrathin cryo-sectioning and imaging under an electron microscope).

In a second embodiment of this invention, phenotypic engineered spores are prepared by a simpler procedure in which living spores suspended in sterile buffer solution are contacted with a particular hydrophobic chemical dissolved in an amphiphilic solvent such as acetone, N,N-dimethylformamide, dimethylsulfoxide, and N,N-dimethylacetamide. For spore engineering, 200 μL of a heat-activated spore suspension is rapidly mixed with 5 μL of a solution containing a selected hydrophobic molecular probe similar to those listed in Table 1, and the mixture is incubated at non-deleterious conditions, for example, at room temperature for 10-15 min with occasional shaking. Alternatively, the mixture may be incubated at 0° C. for 30 minutes. After incubation, the engineered spores are washed twice with a cold sterile aqueous solution and resuspended in a cold aqueous solution.

In a third embodiment of this invention, phenotypic engineered spores are prepared from living spores suspended in sterile, deionized water. The spores are then contacted with a fine emulsion of a hydrophobic molecular probe under conditions that favor apolar (hydrophobic) binding of the selected biochemical to the spores. Fine emulsions of hydrophobic molecular probes may be easily produced as illustrated by the following example using diacetyl fluorescein (DAF) to engineer spores. An emulsion is prepared by mixing 2 mL of an acetone solution containing 0.5 mg/mL DAF with 0.5 mL deionized water, heating the mixture at 100° C. for 3 minutes and cooling it in ice for 5 minutes. For spore engineering, about 10 μL of the emulsion is mixed with about 85 μL of a heat-activated spore suspension and the mixture is incubated at room temperature for about 10 minutes with occasional shaking. After incubation, the spores are washed, generally twice, in cold buffer. The resulting spores can be experimentally shown to have acquired a man-made, fluorogenic functionality placed under control of the germination machinery of the spore. That is, the engineered spores of this invention are not fluorescent by themselves, but rapidly respond to the presence of germinants in their immediate environment by producing bright fluorescent light.

In a fourth embodiment of this invention, phenotypic engineered spores are prepared from microbial spores that have been previously committed to germinate by contacting them to a specific germinant for 1-3 minutes. Commitment is considered a measure of the first irreversible reaction preceding germination and spore outgrowth into a vegetative bacterium (Gordon, S. A. et al. (1981) Commitment of bacterial spores to germinate. Biochem. J. 198:101-106. Setlow, P. (2003) Spore Germination. Curr. Opinion Microbiol. 6: 550-556). Since committed spores behave differently than normal spores in many important respects, phenotypic engineered spores prepared from committed spores can find novel, practical applications in the recent field of spore-based biosensing (U.S. Pat. No. 6,872,539, Rotman). For example, we discovered that committed spores respond differently to environmental signals and also that they have different germinant specificity than normal (not committed) spores. A particularly striking illustration of this discovery is our observation that D-alanine, a well known competitive inhibitor of L-alanine-induced germination for many bacterial spores (Moir, A. and Smith, D. A. 1990. The genetics of bacterial spore germination. Annu. Rev. Microbiol. 44: 531-53), becomes an efficient inducer of germination for committed spores and also for phenotypic engineered committed spores constructed according to this invention.

An embodiment useful for using the invention as biological indicator for sterility testing is to use spores dried in appropriate matrices commonly used in the sterility testing industry such as strips or disks of filter paper. After the spores have been subjected to a sterilization process, they are converted to phenotypic engineered spores directly in the matrix (i.e., in situ). This embodiment is preferred when using phenotypic engineered spores as biological indicators for testing steam-based sterilizers such as autoclaves, that may release molecular probes from the engineered spores.

Some examples of the types of molecular probes suitable for preparing phenotypic engineered spores according to this invention are shown in Table 1. The compounds listed in the table are representative of hydrophobic chemicals suitable for use in the present invention, but are not the only such compounds useful herein. It should also be noted that molecular probes suitable for the invention can have diverse functionalities. For example, some molecules can be enzyme substrates while others can be molecules that become bioluminescent or fluorescent when forming complexes with ions (such as calcium, magnesium, and iron), nucleic acids (such as DNA and RNA), or proteins (such as luciferase). A person of normal skill in the art will be able to determine without too much experimentation the type of molecular probe suitable for constructing phenotypic engineered spores according to this invention.

TABLE 1
Molecular Probes Suitable for Phenotypic Engineering of Spores

| | Engineered Synthetic Functionality |
|---|---|
| Fluorogenic probes | Engineered spores transduce external |
| (e.g., enzyme substrates) | germination signals into fluorescent signals |
| Fluorogenic probes | Engineered spores transduce external |
| (e.g., nucleic acid stains) | germination signals into fluorescent signals |
| | through DNA/RNA binding |
| Fluorogenic probes | Engineered spores transduce external |
| (e.g., calcium probes) | germination signals into fluorescent signals |
| | through calcium binding |
| Chromogenic probes | Engineered spores transduce external |
| (e.g., pH indicators) | germination signals into colored signals |
| Chemoluminescence | Engineered spores transduce external |
| probes | germination signals into chemo-luminescent |
| | signals |
| Bioluminescence probes | Engineered spores transduce external |
| | germination signals into bioluminescent signals |
| Indigogenic probes | Engineered spores transduce external |
| | germination signals into insoluble indigo dyes |
| Quantum Dots | Engineered spores release quantum dots when |
| | exposed to external germination signals |
| Hydrophobic, | Engineered spores release biologically active |
| biologically active | compounds when exposed to external |
| compounds | germination signals |

The usefulness of the present invention is illustrated by the following test for detecting coliform bacteria (the analyte) in a sample. For this practical test, the phenotypic engineered spores are engineered according to the present invention to be fluorogenic by incorporating dipropionylfluorescein in the spores and allowing it to interface with the spore's germination apparatus. The engineered spores are able to detect the analyte because most coliforms have β-D-galactosidase (EC 3.2.1.23), also known as lactase, an enzyme used as a specific marker for fecal contamination of environmental waters. The test system consisted of a buffer solution with the following additions:

(A) Engineered, fluorogenic spores of Geobacillus stearothermophilus.

(B) Lactose, a germinogenic substrate releasing D-glucose (a potent, specific germinant of Bacillus megaterium spores) when hydrolyzed by β-D-galactosidases.

Under appropriate pH and temperature conditions (e.g., pH 6.8-7.8 and 20° C. to 40° C.) coliform bacteria containing β-D-galactosidase produce D-glucose (from lactose hydrolysis) which, in turn, triggers spore germination and concomitant fluorescence due to hydrolysis of dipropionylfluorescein integrated into the spores. The fluorescence produced in the system was measured using standard fluorometry.

The components and reagents for engineering spores according to the present invention may be supplied in the form of a kit in which the simplicity and sensitivity of the methodology are preserved. All necessary reagents can be added in excess to accelerate the reactions. In preferred embodiments, the kit will also comprise a preformed biosensor designed to receive a sample containing an analyte. The exact components of the kit will depend on the type of assay to be performed and the properties of the analyte being tested.

Considering that spores of many diverse organisms have common physical and functional properties, it is expected that the present invention will function well with spores prepared from different spore-forming species including bacteria, fungi, plants, and yeast.

Table 2 lists several spore-forming bacteria and corresponding germinants. It should be noted that mutants of spore-forming organisms in which the specificity of the germinant receptor has been altered can also be engineered using the inventive method.

TABLE 2
Spore forming bacteria and corresponding spore germinants

| Bacteria | Germinant |
|---|---|
| Bacillus atrophaeus | L-alanine |
| Bacillus anthracis | L-alanine + inosine |
| Bacillus cereus | L-alanine + adenosine |
| Bacillus licheniformis | Glucose, Inosine |
| Bacillus megaterium | Glucose, L-proline, KBr |
| Geobacillus stearothermophilus | Complex medium (LB broth) |
| Bacillus subtilis | L-alanine |

Detection. Many of the embodiments of the present invention employ optical detection of spore germination. Detection can be enhanced through the use of spores producing colored, fluorescent, luminescent, or phosphorescent enzymatic products during germination. In a preferred embodiment employing a previously described biosensor (U.S. Pat. No. 6,872,539, Rotman), a charge-coupled device (CCD) readout is used for imaging the response of the system to the analyte in the form of discrete luminescent microwells randomly distributed throughout the biosensor.

### EXAMPLES

The following non-limiting examples provide results that demonstrate the effectiveness of using phenotypic engineered spores for biosensing and sterility testing. All parts and percents are by weight unless otherwise specified.

### Example 1

### Detection of Escherichia coli Containing β-Lactamases

Detection of bacteria containing β-lactamases (EC 3.5.2.6) is clinically important because β-lactamases are usually good markers of bacterial resistance to β-lactam antibiotics. This example illustrates an application of the invention in the LEXSAS™, a biosensing system previously used for detecting low levels of bacteria in near real time (U.S. Pat. No. 6,872,539, Rotman; and Rotman, B. and Cote, M. A. Application of a real-time biosensor to detect bacteria in platelet concentrates. (2003) Biochem. Biophys. Res. Comm., 300:197-200). Using self-reporting, fluorogenic, phenotypic engineered spores in the LEXSAS™ allows the LEXSAS™ to function more efficiently than other systems in which normal spores were used as detectors.
Enzymatic Production of Germinant. In this example, E. coli cells (the analyte) produce L-alanine (the germinant) by cleavage of L-alanyl deacetylcephalothin according to the following reaction: 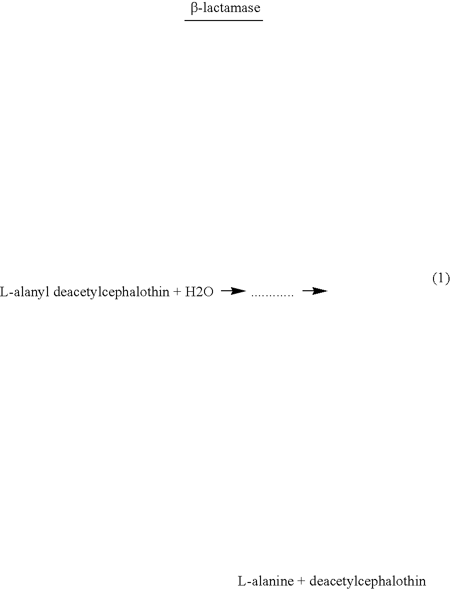
Spores. Spores derived from B. cereus 569H (ATCC 27522), a strain with constitutive β-lactamase II, were used. The spores require mixtures of amino acids and nucleosides for germination, e.g., L-alanine plus adenosine. The spores were obtained by growing bacteria in sporulation agar medium (ATCC medium No. 10) at 37° C. for 1-4 days. The spores were harvested with cold deionized water, heated at 65° C. for 30 min (to kill vegetative cells and to inactivate enzymes) and washed three or more times with deionized water. If necessary, the spores may be further purified according to conventional methodologies such as sonication, lysozyme treatment, and gradient centrifugation (Nicholson, W. L., and Setlow, P. (1990). Sporulation, germination, and outgrowth, p. 391-450. in C. R. Harwood and S. M. Cutting (ed.), Molecular biological methods for Bacillus. John Wiley & Sons, Sussex, England). After spore purification, the spores are resuspended in sterile, deionized water and stored at 4° C. Spore suspensions give satisfactory results after storage at this temperature for up to eight months. Alternatively, the spores may be lyophilized for longer storage.

For phenotypic engineering, about 3×10⁷ spores were first dried under vacuum at room temperature, and then resuspended in 35 μL of acetone containing 1.0 mg/mL dipropionylfluorescein. The spore suspension was stirred for about one minute, and then the acetone was eliminated by evaporation under vacuum at room temperature. The resulting phenotypic engineered spores were resuspended in 100 mM TRIS-20 mM NaCl, pH 7.4, and washed twice in the same buffer.

Reaction mixture. Assays are set up in 96-well microtiter plates. Each well receives 0.18 mL of B. cereus engineered spores (5×10⁷ spores per mL) suspended in 100 mM sodium phosphate buffer, pH 7.2, containing 2 mM adenosine and 50 mM L-alanine deacetylcephalothin, the germinogenic substrate. This substrate is a C10 alanyl ester of deacetylcephalothin liberating L-alanine upon enzymatic hydrolysis of the β-lactam ring according to reaction (1). Synthesis of the substrate has been previously described by Mobashery S, and Johnston M. Inactivation of alanine racemase by β-chloro-L-alanine released enzymatically from amino acid and peptide C10-esters of deacetylcephalothin. (Biochem. 26:5878-5884 (1987)). Test samples (20 μL) containing a bacterial analyte (for example, E. coli K-12 (ATCC 15153) cells) are dispensed into each well, and the plate is incubated at 37° C. The number of tested bacterial cells in the sample may vary from 30 to 10,000. Using a microtiter plate fluorometer, fluorescence (excitation at 488 nm, emission at 520 nm) of individual wells is recorded at zero time and at 2-min intervals. Under these conditions, E. coli cells trigger appearance of fluorescence due to the following interconnected reactions:

(1) E. coli β-lactamase hydrolyses the germinogenic substrate (C10 L-alanyl deacetylcephalothin) liberating L-alanine, which, in turn, induces germination in phenotypic engineered, fluorogenic spores surrounding the E. coli cells;

(2) Germination of the engineered spores promotes release of fluorescent products from the spores;

(3) The course of the reaction is measured fluorometrically.

Appropriate positive and negative controls are included in the test.

### Example 2

### Detection of Pseudomonas aeruginosa by Aminopeptidase Activity

This is another example illustrating the use of the invention in the LEXSAS™. The bacterial analyte is P. aeruginosa (ATCC 10145), a well known human pathogen.

Enzymatic Production of Germinant. In this example, cells of P. aeruginosa (the analyte) have aminopeptidases producing L-alanine (the germinant) by hydrolysis of L-alanyl-L-alanine (Ala-Ala), a germinogenic dipeptide that does not induce spore germination by itself. Aminopeptidases belong to an extended family of enzymes that is present in practically all bacterial species and accordingly are considered universal bacterial markers. The biosensor response to bacterial analytes is based on their generating L-alanine from Ala-Ala according to reaction (2). 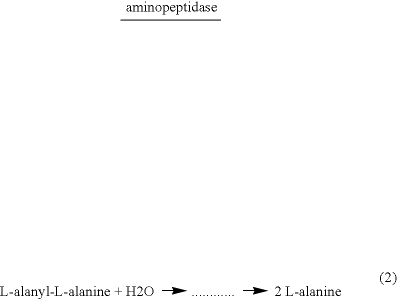
Spores. Spores derived from B. cereus 569H (ATCC 27522) were prepared and engineered as indicated above for Example 1, except that the fluorogenic molecular probe for the engineering was diacetylfluorescein.

Biosensor operation. When using phenotypic engineered spores (constructed according to this invention) in the LEXSAS™, the spores produce fluorescence in response to presence of bacteria, which in this example are cells of P. aeruginosa. Biosensing was performed using glass fiber disks (Whatman GF/A, 6.35 mm diameter) impregnated with a 12-μl volume from a 40-μL reaction mixture containing 4.5×10⁷ phenotypic engineered spores of B. cereus, 100 mM TRIS-20 mM NaCl buffer, pH 7.4, 0.9 mM Ala-Ala, 0.47 mM adenosine (or inosine), and a variable number of P. aeruginosa. Appropriate positive and negative controls were included in the test. The number of P. aeruginosa tested varied from 30 to 10,000 cells per sample. The disks were incubated in a moist chamber at 37° C. for 15 minutes. After incubation, fluorescence images of the disks were captured and quantified using an image analysis system previously described (Rotman, B. and MacDougall, D. E. 1995 Cost-effective true-color imaging system for low-power fluorescence microscopy. CellVision 2:145-150). Disk fluorescence is expressed as “sum of fluorescent pixels” measured inside a square region of 3,600 pixels in the image center. Typical results (Table 3) demonstrate that the LEXSAS™ operating with spores engineered according to this invention performs with a high signal-to-noise ratio.

TABLE 3
Detection of P. aeruginosa in the LEXSAS ™

| Disk Content | Relative Fluorescence(1) | Signal/Noise |
|---|---|---|
| P. aeruginosa | 22,144 ± 1,727 | 14.6 |
| Control (no analyte) | 1,510 ± 108 | |
| Positive control(2) | 28,987 ± 2,175 | |

(1)Average sum of fluorescent pixels per disk ± SD of the mean. Triplicate disks were used per sample.
(2)Phenotypic engineered spores germinated with a mixture of L-alanine and inosine.

### Example 3

### Biological Indicators for Dry Heat Sterility Testing

In this example, the invention was used to monitor dry heat sterilization using preparations of fluorogenic spores of B. atrophaeus (ATCC 9372) engineered as indicated above.

Spores. Spores were derived from B. atrophaeus (ATCC 9372)—a strain commonly used as biological indicators for dry-heat sterilization. Normal spores were prepared as indicated above for Example 1. The spores require L-alanine and inosine for germination. For constructing phenotypic engineered spores, normal spores were heated at 65° C. for 30 min, washed and resuspended in 100 mM Tris-NaCl buffer, pH 7.4. A sample of 200-μL of the spore suspension (in a 1.5-mL polyallomer Beckman tube) was mixed with 5 μL of dimethylsulfoxide (DMSO) containing 5 mg/mL dibutyryl fluorescein as fluorogenic molecular probe. The mixture was incubated at room temperature for 10 minutes, and then the spores were pelleted by centrifugation at 12,000×g for 5 minutes at 4° C. After removing the supernatant, the pellet was resuspended with 200 μL of buffer. The suspension was transferred to a new polyallomer tube and the spores were washed twice with sterile deionized water.

Biological indicator. To use the phenotypic engineered spores as biological indicators, about 3×10⁶ spores were dried on glass fiber discs (Whatman GF/A, 6.35 mm diameter). The disks were exposed to dry heat at temperatures ranging from 140° C. to 160° C. for variable periods of time. After the sterilization process, spore germination was tested by adding 12 μL of Luria broth (the germinant) to each disk, and incubating the disks in a moist chamber for 20 minutes at 37° C. After incubation, fluorescence images of the disks were captured using an image analysis system for measuring fluorescence of solid materials (Rotman, B. and MacDougall, D. E. (1995). Cost-effective true-color imaging system for low-power fluorescence microscopy. CellVision 2:145-150). The results shown in Table 4 demonstrate that the phenotypic engineered spores performed well as biological indicators because spores in discs exposed to inadequate sterilization conditions (e.g., 150° C. for 12 minutes) retained partial ability to release fluorescent products in response to germination signals. Moreover, the data from this and other similar experiments indicate that biological indicators made of phenotypic engineered spores have D values comparable to that of normal spores.

TABLE 4
Dry Heat Sterility Testing

| Time (min) | Relative Fluorescence(1) | % “Killing” |
|---|---|---|
| 0 | 62,344 ± 12,456 | 0 |
| 4 | 24,736 ± 1,957 | 60 |
| 8 | 11,796 ± 5,844 | 81 |
| 12 | 4000 ± 1946 | 94 |
| Dead Spores(2) | 0 | 100 |

(1)Average sum of fluorescent pixels per disk ± SD of the mean. Triplicate disks were used for each sample.
(2)Spores were killed by exposing disks to dry heat at 150° C. for 66 minutes.

### Example 4

### Biological Indicators for Steam Heat Sterility Testing Constructed by in situ Engineering of Spores

In this example, this invention was used to construct in situ biological indicators for steam heat sterility testing.

Spores. Spores were derived from G. stearothermophilus (ATCC 12980)—a strain commonly used as biological indicators for steam-heat sterilization. Normal spores were prepared as indicated above for Example 1. The spores were germinated in the presence of Luria broth (LB).

Biological indicator. About 1×10⁶ spores suspended in 0.5 μL of sterile deionized water were dried as a small spot on a rectangular strip of glass fiber paper (Whatman GF/A) 6×17 mm. After drying, the strip was exposed to steam heat in an autoclave (VWR Accusterilizer) set at 121° C. for variable periods of time. After sterilization, the spores on the strip were converted to phenotypic engineered spores by adding 20 μL of 100 mM TRIS-20 mM NaCl, pH 7.4 buffer containing 32 μM dibutyryl fluorescein and 70.4 mM dimethylsulfoxide (DMSO). The strip was incubated at room temperature for 5 minutes, and then it was placed in a small glass container for development by lateral flow diffusion of a germinant solution for 30 minutes at 55° C. The germinant solution was Luria broth (LB) diluted 1:7 in 100 mM TRIS-20 mM NaCl buffer, pH 7.4 enriched with 112 mM L-alanine. After development, fluorescence images of the strips were captured using an image analysis system for measuring fluorescence of solid materials (Rotman, B. and MacDougall, D. E. (1995). Cost-effective true-color imaging system for low-power fluorescence microscopy. CellVision 2:145-150). The data shown in Table 5 demonstrate that phenotypic engineered spores constructed directly on a paper strip perform satisfactorily as biological indicators. That is, the engineered spores are still capable of germinating and producing fluorescence after exposing them to an inadequate steam heat process (e.g., 2.5 minutes), but do not produce fluorescence after a 100% lethal sterilization process. The D value of phenotypic engineered spores killed by steam sterilization was found to be similar or higher than that of normal spores, i.e., between 2 and 3 minutes.

TABLE 5
Phenotypic engineered spores as biological indicators for steam heat

| Time (min) | Relative Fluorescence(1) | % “Killing” |
|---|---|---|
| 0 | 65,084 ± 31,231 | 0 |
| 15 | 0 ± 0 | 100 |

(1)Average sum of fluorescent pixels per disk ± SD of the mean. Duplicate strips were used for each sample.

### Example 5

### Using Phenotypic Engineered for Cell-based Biosensing of Biological Warfare Agents

There is an urgent need for new technology capable of monitoring the environment for biological warfare agents in near real time. In this example, spores engineered according to the invention are used as living detecting components of a rapid cell-based biosensor for biological warfare agents. As in Example 1, the biosensor operates via the LEXSAS™ except that in this case the analytes are not bacteria but biological warfare agents tagged with a germinogenic enzyme. For example, a target biological warfare agent—such as Staphylococcus enterotoxin B—can be tagged with a specific antibody covalently linked to alkaline phosphatase to become a suitable analyte.

Spores. Normal spores derived from B. megaterium (ATCC 14581) were prepared as indicated for Example 1, and subsequently phenotypic engineered as indicated for Example 3 except that Syto 9 (InVitrogen) was used as fluorogenic molecular probe. Syto 9 is a nucleic acid stain that increases its fluorescence about 50 times when contacted with either DNA or RNA (Haugland, R. P. 2005 The Handbook—A Guide to Fluorescent Probes and Labeling Technologies.—Molecular Probes, Eugene, Oreg., 10th edition). These spores are germinated specifically by monosaccharides such as D-glucose, D-fructose, D-mannose, and methyl β-D-glucopyranoside. When using B. megaterium spores in the LEXSAS™, suitable germinogenic substrates are, for example, lactose (hydrolyzed by β-galactosidases), sucrose (hydrolyzed by sucrase), glucose-1-phosphate and glucose-6-phosphate (both hydrolyzed by phosphatases).

Biosensor operation. Spores of a non-virulent strain of B. anthracis (Sterne strain) were used as subrogates of spores causing anthrax. The spores were first coated with a specific anti-B. anthracis rabbit IgG, and then captured on paramagnetic beads coated with protein A. After separating, washing and blocking the magnetic beads with normal goat IgG, the spores on the beads were exposed to a secondary specific anti-B. anthracis goat IgG labeled with alkaline phosphatase. This process of using two specific antibodies (or other ligands) binding different epitopes for capturing and tagging biological particles is often used to enhance selectivity of a test and also to reduce the baseline noise, and it is critical for achieving high levels of selectivity necessary to avoid false positives. At the end of the process, the phosphatase-labeled beads are magnetically separated and then introduced in a biosensor capable of detecting and quantifying individual magnetic beads. The biosensor is a passive microfluidic device fabricated by spin coating a 15-μm thick silicon nitride photoresist on a 13-mm diameter polycarbonate filter membrane with uniform 0.2 μm pores. Subsequently, the silicon layer is photolithographically etched to produce about 80,000 MICRO-COLANDER® diagnostic analyzers. A MICRO-COLANDER® analyzer is a microscopic reaction chamber of five-picoliter (5×10⁻¹² L) volume that drains through thousands of uniform pores located at the bottom of the chamber (U.S. Pat. No. 6,872,539, Rotman). Consequently, the biosensor performs as a filtration and collection device for capturing, detecting and enumerating weaponized biological particles (WPBs). The fact that each MICRO-COLANDER® analyzer functions as an independent biosensor provides for both single magnetic bead sensitivity and straight forward quantitative analysis because the number of fluorescent pores of the MICRO-COLANDER analyzer containing WBPs equals the number of WBPs in the sample. Fluorescent images of the biosensor collected and analyzed at time intervals provide quantitative data.

What is claimed is:

1. Phenotypically engineered spore that includes a man-made functionality under the control of the spore's natural germination apparatus to give the spore self-reporting capability.

2. The spores of claim 1, wherein the man-made functionality is introduced by contacting the spores with a hydrophobic compound.

3. The spores of claim 2, wherein the hydrophobic compound is selected from compounds which have a property selected from the group consisting of fluorogenicity, chromogenicity, chemiluminogenicity, bioluminogenicity, and indigogenicity.

4. The spores of claim 3, wherein the engineered spores do not have the property of being fluorescent, colored, chemiluminescent, bioluminescent, or producing insoluble colored pigments by themselves but acquire one of these properties in response to the presence of a germinant for the spores in their immediate environment.

5. The spores of claim 2, wherein the spores are selected from the group consisting of bacteria, fungi, plants, and yeast.

6. Phenotypic engineered spores that are self-reporters of germination.

7. Use of the phenotypically modified microbial spores of claim 1 as self-indicators of adequate sterility of a system.

8. The use of claim 7, wherein the spores act as both signal-sensors and signal-transducers of analyte specific signals.

9. A sterility-indicating kit comprising a preformed biosensor which includes the spores of claim 1 designed as self-indicators of sterility of a system.

10. Use of the phenotypically modified microbial spores of claim 1 as self-indicators of biological warfare agents.

11. A method of preparing the phenotypically engineered spores of claim 1 comprising suspending living spores in a liquid, contacting the spores with a hydrophobic compound under conditions which cause the hydrophobic compound to incorporate and self-assemble in the spores to form modified spores, and recovering the modified spores.

12. The method of claim 11, wherein the living spores are dried living spores containing less than about 5% extracellular water and are suspended in a non-aqueous liquid containing a hydrophobic molecule to form a spore suspension, the spore suspension is incubated for a sufficient period of time to allow the incorporation and self-assembling of the hydrophobic molecule into the spores, and removing the non-aqueous liquid.

13. The method of claim 12, wherein the non-aqueous liquid is removed under vacuum.

14. The method of claim 12, wherein the dried living spores contain less than about 1% extracellular water.

15. The method of claim 12, wherein the dried living spores containing less than 5% extracellular water are prepared by heat-activating a spore suspension in sterile deionized water at a temperature of about 50 to 110° for about 5 to 60 minutes, spinning the heat-activated suspension to pellet the spores and form a supernatant, removing the supernatant, and drying the pellets under vacuum over a desiccant.

16. The method of claim 12, wherein the non-aqueous liquid is selected from the group consisting of acetone, acetonitrile, ethyl acetate, methyl ethyl ketone, tetrahydrofuran, and toluene.

17. The method of claim 11, wherein dried living spores are suspended in a sterile buffer solution to form a suspension, a hydrophobic compound is dissolved in an amphiphillic non-aqueous solvent to form a solution, the suspension and the solution are combined and incubated to form the phenotypically modified spores, recovering the modified spores, and resuspending the modified spores in a sterile aqueous solution.

18. The method of claim 17, wherein the amphiphillic non-aqueous solvent is selected from the group consisting of acetone, N,N-dimethylformamide, dimethylsulfoxide, and N,N-dimethylacetamide.

19. The method of claim 17, wherein the incubation occurs at about room temperature for about 5 to 25 minutes.

20. The method of claim 17, wherein the incubation occurs at about 0° C. for about 30 minutes.

21. The method of claim 11, wherein dried living spores are suspended in sterile deionized water to form an aqueous suspension, preparing an emulsion of a hydrophobic compound by dispersing an organic solution containing the hydrophobic compound in an aqueous solution, combining the suspension and the emulsion and allowing them to incubate to form the phenotypic modified spores.

22. The method of claim 11, wherein microbial spores have been committed to germinate by previous contact with a germinant.

23. The method of claim 22, wherein the spores have been committed to germinate by contact of about 1 to 5 minutes with a germinant.

24. The method of claim 23, wherein after contact with the germinant, the committed spores are spun to pellet and form a supernatant, removing the supernatant, and resuspending the committed spores in a sterile aqueous solution.
